(19) United States
(12) Patent Application Publication
(10) Pub. No.: US 2002/0182607 A1
(43) Pub. Date: Dec. 5, 2002

(54) Compositions and methods for parsing gene structure

(76) Inventor: Shankar Subramaniam, La Jolla, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/882,359

(22) Filed: Jun. 14, 2001

Related U.S. Application Data
(60) Provisional application No. 60/295,222, filed on May 31, 2001.

(51) Int. Cl. C12Q 1/68; C07H 21/04

(52) U.S. Cl. 435/6; 536/24.3

(57) ABSTRACT

The invention provides a method for determining a sequence boundary. The method includes the steps of (a) contacting a population of addressed fragments of eukaryotic genomic DNA with a target polynucleotide, the target polynucleotide binding a terminal sequence of a DNA region, the addressed fragments of eukaryotic genomic DNA being at least 100 nucleotides in length; (b) determining a relative order for 2 or more of the addressed fragments compared to a sequence of the genomic DNA; (c) identifying a pair of fragments among the 2 or more addressed fragments that alternatively bind the terminal sequence of a region; and (d) determining for the sequence of the genomic DNA a relative location of a boundary of the region compared to a location of at least one genomic DNA fragment in the pair.

This application is based on, and claims the benefit of, U.S. Provisional Application No. 60/295,222, filed May 31, 2001, and entitled COMPOSITIONS AND METHODS FOR PARSING GENE STRUCTURE, and which is incorporated herein by reference.

### BACKGROUND OF THE INVENTION

This invention relates to polynucleotide sequence analysis and, more specifically to identifying functional regions of genomic DNA.

The recent expansion in the amount of genetic sequence information available in public and private databases is causing a change in perspective for biological and biomedical research. Where previously phenomena were observed and comparisons made between biological systems, currently, comparison between sequences is being carried out in the hope of identifying phenomena such as structure and function. It has long been a fundamental principle of biology that the sequence of a gene determines its structure and its function. However, due to incomplete knowledge of the rules which correlate sequence with structure and function, determination of function based on sequence analysis alone is not yet available. As a result, biological and biomedical research rely on empirical methods to determine the function of genes.

In addition, the availability of sequences for entire genomes is broadening perspective to include not only the function of single genes but the function of sets of genes or even entire genomes. Thus, not only is the wealth of sequence data overwhelming the capacity of traditional empirical methods to identify functions for the individual genes being identified, but the number of genes to be analyzed in observing an entire genome is also beyond the capacity of traditional methods.

In the relatively few cases where a correlation between sequence and structure or function has been identified errors in the sequences stored in databases can severely compromise the ability of sequence analysis to identify functional regions. A variety of errors are known to reside in the sequences stored in databases. For example, Genbank, a commonly used public sequence database, is estimated to contain errors in 2% of its sequences. Errors include, for example, sequence additions, deleted sequences and incorrect sequences. Such errors can occur at a variety of stages in the determination and manipulation of sequence data including, for example, sequencing artifacts and data management errors. In addition databases can contain annotation errors which frustrate efforts to identify correlations between sequence and structure or function. In this regard, Genbank has been estimated to contain annotation errors in 15% of its entries including, for example, erroneous identification of the source organism or chromosome of a sequence or identification of a partial gene sequence as a complete gene.

In many cases errors go unnoticed. In cases where errors are eventually corrected in a database the damage can be difficult to reverse as errors are proliferated in the scientific literature. Sequence errors can have devastating effects on a research program such as those aimed at developing therapeutics and diagnostics intended for use in humans. Identification and correction of errors in a database can be difficult requiring, for example, re-evaluation of primary literature, re-analysis of raw data, or even repetition of the sequencing effort initially used to produce the data.

Thus, there exists a need for methods to identify functional regions within large collections of sequence data. A need also exists for methods to evaluate genomic DNA sequences for accuracy. The present invention satisfies these needs and provides related advantages as well.

### SUMMARY OF THE INVENTION

The invention provides a method for determining a sequence boundary. The method includes the steps of (a) contacting a population of addressed fragments of eukaryotic genomic DNA with a target polynucleotide, the target polynucleotide binding a terminal sequence of a DNA region, the addressed fragments of eukaryotic genomic DNA being at least 100 nucleotides in length; (b) determining a relative order for 2 or more of the addressed fragments compared to a sequence of the genomic DNA; (c) identifying a pair of fragments among the 2 or more addressed fragments that alternatively bind the terminal sequence of a region; and (d) determining for the sequence of the genomic DNA a relative location of a boundary of the region compared to a location of at least one genomic DNA fragment in the pair.

### DETAILED DESCRIPTION OF THE INVENTION

This invention provides methods for identifying sequence boundaries in genomic DNA. The methods can be used to determine which regions of genomic DNA are included in an expressed polynucleotide. For example, the methods can be used to identify exons that are included in an expressed polynucleotide. An advantage of the invention is that the methods can be used with a population of expressed polynucleotides to simultaneously determine boundaries for a plurality of regions included in the population of expressed polynucleotides. Thus, differential expression of exons can be efficiently determined to map genes, determine tissue specific expression patterns, or identify polymorphisms in individuals.

The invention also provides methods for identifying a boundary in a polynucleotide sequence. For example, the methods can be used to identify a sequence boundary for a region of a genomic DNA sequence. Identification of sequence boundaries can be useful in identifying a variety of regions in a genome including, for example, an operon, cistron, gene, open reading frame, transposon, untranslated region, coding sequence such as an exon, non-coding sequence such as an intron, or expression element such as a promoter or terminator. An advantage of the invention is that the methods can be used to identify regions of a genomic DNA in cases where regions have not been or can not be identified based on sequence analysis alone. The methods can also be used to identify regions in cases where an error present in a sequence database precludes identification of a region by sequence analysis.

As used herein the term “boundary,” when used in reference to a polynucleotide sequence, refers to a location at which a region of polynucleotide sequence begins or ends. The location can be identified as one or more nucleotides in a sequence including, for example, an initial nucleotide or final nucleotide of a region. The location can also be identified relative to one or more nucleotides in a region including, for example, a location between a final nucleotide of one region and an initial nucleotide of an adjacent region.

As used herein the term “terminal sequence” refers to the order of nucleotides at the 5′ or 3′ end of a region of a polynucleotide. A terminal sequence can be at a physical terminus of a polynucleotide molecule or within a polynucleotide molecule. The term includes any number of nucleotides sufficient to identify the end of a region of a polynucleotide including, for example, 2 nucleotides to tens to even hundreds of nucleotides. Thus, a terminal sequence can include 2 or more nucleotides, 4 or more nucleotides, 6 or more nucleotides, 8 or more nucleotides, 10 or more nucleotides, 20 or more nucleotides, 30 or more nucleotides, 40 or more nucleotides, 50 or more nucleotides or 100 or more nucleotides.

As used herein the term “region,” when used in reference to DNA, refers to a continuous sequence of DNA having natural or assigned boundaries. Natural boundaries in a DNA sequence are locations at which a component of a cell differentiates or separates a continuous sequence in the DNA from another sequence. A natural boundary can differentiate or separate a variety of continuous sequences of DNA from another sequence including, for example, an operon, cistron, gene, open reading frame, transposon, untranslated region, coding sequence such as an exon, non-coding sequence such as an intron, translated sequence, transcribed sequence, or expression element such as a promoter or terminator. Assigned boundaries in a DNA sequence are locations at which a continuous sequence in the DNA can be differentiated from another sequence according to an identified structural or functional property. Assigned boundaries can differentiate or separate a variety of continuous sequences of DNA from another sequence including, for example, a sequence having a natural boundary, a sequence that is homologous to a second sequence, a sequence that is repeated, or a sequence that corresponds to a previously identified sequence.

As used herein the term “genomic DNA” refers to a chromosomal polymeric deoxyribonucleotide molecule occurring naturally in a cell and containing sequences that are not transcribed into RNA by the cell. A chromosomal polymeric deoxyribonucleotide molecule of a eucaryotic cell contains at least one centromere, two telomeres, one origin of replication, and one sequence that is not transcribed into RNA by the eucaryotic cell including, for example, an intron or transcription promoter. A chromosomal polymeric deoxyribonucleotide molecule of a procaryotic cell contains at least one origin of replication and one sequence that is not transcribed into RNA by the procaryotic cell including, for example, a transcription promoter. A eucaryotic genomic DNA can be distinguished from a procaryotic genomic DNA, for example, according to the presence of introns in eucaryotic genomic DNA and absence of introns in procaryotic genomic DNA.

As used herein, the term “fragment,” when used in reference to a genomic DNA refers to a polymeric deoxyribonucleotide molecule having identical nucleotide sequence to a contiguous portion of genomic DNA. The term is intended to include a contiguous portion of a polymeric deoxyribonucleotide molecule isolated from an organism or produced from a polymeric deoxyribonucleotide molecule isolated from an organism. A produced polymeric deoxyribonucleotide molecule includes those obtained by any method known in the art for obtaining a replica of a polymeric deoxyribonucleotide molecule including, for example, DNA template directed synthesis such as the polymerase chain reaction (PCR) or synthesis based on a determined sequence such as solid phase synthesis techniques.

As used herein, the term “addressed” when used in reference to a DNA fragment in a population refers to a DNA fragment that is bound to a substrate such that the DNA fragment can be distinguished from others in the population according to a property of the bound substrate. A bound substrate that can be used to distinguish a genomic DNA fragment includes, for example, a particle or a fixed location on a surface or in a volume.

A particle that can be used as a bound substrate includes, for example, silica based particles such as glass beads or particles of polymeric composition such as polyethylene glycol, agarose or SEPHAROSE™. Properties of a particle that can be used to distinguish an addressed genomic DNA fragment from others in a population include, for example, location in a capillary, location in an array or a physical or chemical property unique to the particle. A physical or chemical property of a particle can be imparted by a bound atom or molecule including, for example, a chromophore, fluorophore, or spin label. An example of a particle that can be specifically modified is an encoded chip which can be written or read by high-frequency signals as described for example in Balkenhohl et al., Anqew Chem. Int. Ed. Enal. 35:2288-2337 (1996).

A surface upon which a DNA fragment can be bound and located includes, for example, glass, silicon, silica, paper, nitrocellulose, or polymers such as polyvinylidine difluoride or plastics. A volume in which a DNA fragment can be bound and located includes, for example, gels such as polyacrylamide or agarose. A surface bound DNA fragment can be distinguished from others in a population, for example, according to coordinates identifying its location.

As used herein the term “polynucleotide” refers to a polymer of nucleotide units. The term is intended to include naturally occurring polymers such as polydeoxyribonucleic acid (DNA) and polyribonucleic acid (RNA) and analogs thereof. Examples of naturally occurring DNA include genomic DNA (gDNA), copy DNA (cDNA) and extragenomic DNA such as non-chromosomal plasmids and vectors. Naturally occurring RNA can be, for example, messenger RNA (mRNA), transfer RNA (tRNA) or ribosomal RNA (rRNA). Analogs include polymers that contain one or more non-naturally occurring nucleotide or those that are attached by linkers other than phosphodiester bonds. Examples of a linkage that can occur in an analog include, for example, a phosphorothioate, phosphorodithioate, phosphoramidate, methylphosphonate, phosphorotriester, chiral methyl phosphonate, boranophosphate, or peptide. A polynucleotide is understood to contain any number of nucleotides greater than 2 including, for example, a few, tens, hundreds, thousands or more.

As used herein, the term “target,” when used in reference to a polynucleotide, refers to a polynucleotide that binds to a genomic DNA molecule with sequence dependent specificity. A polynucleotide target need not contain the exact complementary sequence of the polynucleotide to which it binds, so long as binding is specific. Specific binding is understood to be association between two nucleic acid sequences by Watson-Crick hydrogen bonds between nucleotides. Specific binding includes hybridization that occurs in moderate and high stringency conditions as described further below. Target polynucleotides include, for example, expressed polynucleotides or polynucleotides encoding expressed polynucleotides. Expressed polynucleotides include any polynucleotide that is naturally replicated or transcribed from a genomic DNA including, for example, RNA, mRNA, tRNA, or ribosomal RNA. Polynucleotides encoding expressed polynucleotides include polynucleotides having substantially the same sequence or complimentary sequence as a polynucleotide that is naturally replicated or transcribed from a genomic DNA including, for example, post transcriptionally processed or modified products. Examples of polynucleotides encoding expressed polynucleotides include, for example, an RNA, mRNA, tRNA, ribosomal RNA, DNA, exon, genomic DNA fragment cDNA, or analog thereof. Polynucleotide analogs include any polymer having pyridine or pyrimidine bases capable of making sequence specific hybrids with a polynucleotide including, for example, protein nucleic acids.

As used herein the term “alternatively,” when used in reference to hybridization of a target polynucleotide to a pair of DNA fragments, refers to specific binding to one member of the pair and absence of specific binding to the second member of the pair.

The invention provides a method for determining a sequence boundary. The method includes the steps of (a) contacting a population of addressed fragments of eukaryotic genomic DNA with a target polynucleotide, the target polynucleotide binding a terminal sequence of a DNA region, the addressed fragments of eukaryotic genomic DNA being at least 100 nucleotides in length; (b) determining a relative order for 2 or more of the addressed fragments compared to a sequence of the genomic DNA; (c) identifying a pair of fragments among the 2 or more addressed fragments that alternatively bind the terminal sequence of a region; and (d) determining for the sequence of the genomic DNA a relative location of a boundary of the region compared to a location of at least one genomic DNA fragment in the pair.

The methods of the invention can be used to identify a boundary of a region of DNA within a sequence of genomic DNA according to differential binding of a target polynucleotide to sequences flanking either side of the boundary. The target polynucleotide contains a sequence that is complementary to a terminal sequence of the region but lacks complementarity with the sequence flanking the other side of the boundary. Thus, the target polynucleotide preferentially binds the terminal sequence of the region. The location in the genomic DNA at which differential binding occurs is determined in a population of addressed fragments of the genomic DNA. The fragments of genomic DNA provide separation of sequences flanking the boundary such that differential binding can be readily identified on a fragment by fragment basis. Because the fragments are addressed, they can be ordered with respect to the genomic DNA sequence and differentially bound fragments that have adjacent sequences in the genomic DNA identified. The location of a boundary can then be identified in the genomic DNA sequence as residing between the adjacent sequences.

The invention further provides a method for determining a plurality of sequence boundaries. The method includes the steps of (a) contacting a population of addressed fragments of eukaryotic genomic DNA with a target polynucleotide, the target polynucleotide binding a plurality of terminal sequences of DNA regions, the addressed fragments of eukaryotic genomic DNA being at least 100 nucleotides in length; (b) determining a relative order for 2 or more of the addressed fragments compared to a sequence of the genomic DNA for a plurality of sets of 2 or more genomic DNA fragments; (c) identifying a plurality of pairs of fragments among the plurality of sets of 2 or more addressed fragments, the pairs comprising fragments that alternatively bind the terminal sequences of regions; and (d) determining for the sequence of the genomic DNA relative locations of boundaries for a plurality of the regions compared to locations of at least one genomic DNA fragment in each of the pairs.

The methods of the invention can be performed to determine a boundary for one or more genetic regions of genomic DNA from any organism including a procaryote or eucaryote. A sequence boundary can be determined by the methods of the invention for genomic DNA from a eucaryote including, for example, a mammal, such as a human, horse, dog, cow, cat, mouse, rat, pig, or sheep; plant, such as tobacco, A. thaliana, oat, corn, or rice; vertebrate, such as a bird, xenopus laevis or zebrafish; invertebrate, such as D. melanogaster or C. elegans; or microorganism, such as S. pombe or S. cerevisiae. A procaryotic genomic DNA used in the methods of the invention can be from a bacteria, such as E. coli, or Staphylococcus; or an archaea. The methods can also be used to identify a sequence boundary in a genomic DNA from a virus, such as hepatitis C virus or HIV.

Genomic DNA can be isolated from a tissue sample or organism by lysing cells, extracting DNA and purifying the DNA according to methods well known in the art including, for example, those described in Sambrook and Russel, Molecular Cloning: A Laboratory Manual, 3rd ed., Cold Spring Harbor Press, Cold Spring Harbor (2001), and in Ausubel et al., Current protocols in Molecular Biology, John Wiley and Sons, Baltimore, Md. (2000). For multicullular organisms, genomic DNA can be isolated from virtually any cell type, fluid or biological tissue. Convenient sources of genomic DNA from animals include, for example, whole blood, semen, saliva, tears, urine, fecal material, sweat, buccal, skin and hair.

Genomic DNA can be used directly in the methods of the invention or converted into a genomic DNA library prior to use. Methods for generating a genomic DNA library are well known in the art and are described for example in Sambrook and Russel, supra and Ausubel et al., supra. In addition the methods of the invention can be performed with genomic DNA from a previously constructed genomic DNA library including, for example, the mega YAC library created at the Center d'Etude Polymorphisme Humaine CEPH as described in Chumakov et al., Nature 359:380-387 (1992) or those supplied by commercial entities such as InVitrogen (Carlsbad, Calif.) or Research Genetics (Huntsville, Ala.). Genomic DNA libraries and methods of their construction are known in the art including, for example, the C. elegans genomic DNA library produced by the C. elegans sequencing consortium, Science 282:2012 (1998), the human genome library described in Venter et al., Science 291:1304-1351 (2001), and the Drosophila genome library described in Adams et al., Science 287:2185-2195 (2000), each of which has been used to determine the genomic sequences of the respective organisms.

Genomic DNA libraries can be conveniently contained in a variety of vectors and host cells known in the art. An appropriate library format can be chosen by one skilled in the art based on desired properties such as insert size, host, or vector composition. Examples of libraries having different properties and available in the art include, for example, Yeast Artificial Chromosome (YAC) libraries, Cosmid libraries, Bacterial Artificial Chromosome (BAC) libraries and PAC libraries. A YAC contains functional elements of a eukaryotic chromosome and can be replicated in Saccharomyces cerevisiae. YAC vectors allow for the routine cloning of 500 kilobase genomic DNA fragments and can incorporate fragments in the range of 100-1000 kilobases (Burke et al. Science 236:806-812 (1987). An example of a YAC based library useful in the invention and methods for producing a genomic YAC library are described by the C. elegans sequencing consortium, supra (1998). Cosmid vectors can be maintained in Escherichia coli and contain particular X sequences that direct insertion of DNA particles into phage. Cosmid vectors typically accommodate DNA fragments of 35-50 kilobases (Ausubel, supra). BAC vectors are based on Escherichia coli and its single-copy plasmid F factor. BAC vectors have been shown to accommodate inserts as large as 300 kilobases and are routinely constructed with inserts in the range of 100-150 kilobases (Shizuya et al. Proc. Nat. Acad. Sci. USA 89:8794-8797 (1992)). Examples of BAC based libraries useful in the invention and methods for producing a genomic BAC library are described in Venter et al., supra (2001), and Adams et al., supra (2000). PAC vectors including, for example, pPAC4 and pCYPAC2 are maintained in E. coli, are based on the bacteriophage P1 cloning system and can be used to propagate DNA inserts of up to about 95 kilobases (Pierce et al. Proc. Nat. Acad. Sci. USA 89:2056-2060 (1992)).

Vectors which harbor inserts that are smaller or larger than those described above can also be used in the methods of the invention. For example, an individual fragment of genomic DNA from a library can be isolated and fragmented to produce a sub-library. The fragments in the sub-library can be smaller than the fragments in the original library such that they can be manipulated in different vectors having a smaller insert capacity. Thus, any vector known in the art that is suitable for propagation in a prokaryotic or eukaryotic host cell can be useful in the methods of the invention.

A genomic DNA library used in the methods of the invention can contain a complete sequence of an organism's genome or a portion thereof. A portion of a genome can be a contiguous region including, for example, a particular chromosome, contig, locus or gene cluster such as the major histocompatibility locus. Additionally, a library can contain fragments encoding sequences identified by genetic criteria such as association with a certain trait or characteristic. Genetic criteria that can be used to identify a portion of a genomic DNA to be present in a library include, for example, linkage analysis and linkage disequilibrium as described in Haines and Pericak-Vance, Approaches to Gene Mapping in Complex Human Diseases (1998) Wiley-Liss, New York. A library of genomic DNA fragments, such as a library constructed to contain genetically identified sequences, can have fragments from non-contiguous portions of a genome.

A population of eucaryotic genomic DNA fragments can be enriched for, or selectively contain, exon sequences. For example, a library can be enriched for euchromatin, which is the region of a chromosome that contains a majority of active genes, by selectively minimizing or excluding heterochromatin, which is a condensed region of a chromosome that is gene deficient. Euchromatin enriched libraries are known in the art and can be constructed as described, for example, in Venter et al., supra (2001), and Adams et al., supra (2000). An advantage of using an enriched library is that the amount of genomic DNA sequence to be analyzed can be reduced. For example, the Drosophila genome is about 180 Mb in size, one third of which is centric heterochromatin. A population of Drosophila genomic DNA fragments that is selectively enriched for euchromatin can be made to cover 120 Mb of the genome, thereby reducing the size or amount of fragments used to determine boundary regions of genes or exons compared to that required for similar coverage of the full genome.

Genomic DNA fragments can be obtained by amplification from a genome or genomic DNA library using methods such as PCR, and the like. Amplification of a genomic DNA, can be achieved by methods known to those skilled in the art. The polymerase chain reaction (PCR) involves template-dependent extension using thermally stable DNA polymerase and oligonucleotide primers complementary to regions of the sequence to be amplified; extension products incorporating primers then become templates for subsequent amplification steps. (Mullis et al., Cold Spring Harbor Symp. Ouant. Biol. 51:263-273 (1986); Erlich et al., EP 50,424; EP 84,796, EP 258,017, EP 237-362; Mullis, EP 201,184; Mullis et al., U.S. Pat. No. 4,683,202; Erlich, U.S. Pat. No. 4,582,788; and Saiki et al., U.S. Pat. No. 4,683,194). Reviews of the polymerase chain reaction are provided by Mullis, K. B., (supra); Saiki, R. K. et al., (Bio/TechnoloQy 3:1008-1012 (1985)); and Mullis, K. B. et al. (Meth. Enzymol. 155:335-350 (1987)). Other nucleic acid amplification procedures can be used and include self-sustained sequence replication (Guatelli et al., Proc Natl. Acad. Sci. USA 87:1874 (1990)) and ligation-based amplification systems (Wu, D. Y. et al., Genomics 4:560 (1989)).

The amplification methods described above can be performed in a manner to obtain a population of genomic DNA fragments having a determined average size, minimum size or maximum size. For example, a library having genomic DNA inserts of a uniform average size can be amplified using primers to vector sequences flanking the insert to obtain fragments of uniform average size. Fragments of a determined size can also be obtained by direct amplification from genomic DNA and digestion using methods described below.

Isolated or amplified genomic DNA can be fragmented by digestion with an endonuclease. Endonucleases useful in the methods of the invention include those that cleave at a specific recognition sequence and those that non-specifically cleave DNA. Endonuclease are available in the art and can be obtained, for example, from commercial sources such as New England BioLabs (Beverley, Mass.) and Life technologies Inc. (Rockville, Md.). Specific endonucleases can be used to generate polynucleotide fragments of an average size according to the frequency with which the enzyme is expected to cut a random sequence. For example, an endonuclease having a six nucleotide recognition sequence would be expected to produce, on average, fragments that are 4096 base pairs long. Average fragment length can be estimated by treating the DNA as a random sequence and estimating the frequency of a recognition site in the random sequence according to the relationship 4ⁿ=s where n is the number of bases recognized by the endonuclease and s is the average size of the fragments produced. Incubation conditions can also be modified, as described below, to alter the enzymatic efficiency of the endonuclease, thereby altering the average size of the fragments produced. Using the example of an endonuclease having a 6 basepair recognition site, a decrease in enzymatic efficiency can produce fragments that are on average larger than 4096 base pairs long.

Non-specific endonucleases can also be used to produce polynucleotide fragments of a desired average size. One skilled in the art knows that the endonuclease reaction is bimolecular such that the rate of fragmentation by an endonuclease can be manipulated by altering conditions such as the concentrations of the endonuclease, DNA recognition sequence or both. Specifically, a reduction in the concentration of either endonuclease, DNA recognition sequence or both can be used to reduce reaction rate resulting in increased average fragment sizes. Increasing concentrations of either endonuclease, DNA recognition sequence or both will allow for increased efficiency, approaching maximum velocity (Vₘₐₓ)for the particular enzyme leading to reduced average fragment sizes. Other reaction conditions can also affect the rate of cleavage including, for example, temperature, salt concentration and time of reaction. Methods for altering nuclease reaction rates to produce polynucleotide fragments of determined average size are described for example in Sambrook and Russell, supra and Ausubel, supra.

Thus, the methods can use authentic genomic DNA fragments produced from the genomic DNA of an individual. Authentic genomic DNA fragments include those with greater than 98% sequence homology to an individual including, for example, those with greater than 99% sequence homology to an individual, or those with greater than 99.5% sequence homology to an individual. An addressed population of genomic DNA fragments can also include synthetic fragments produced by de novo synthesis based on a reference sequence of genomic DNA or a portion thereof. Such reference sequences can be obtained from a variety of sources including, for example, academic publications, Genbank or a variety of commercial genome sequence databases known in the art. The reference sequence can be that of an individual, population or subgroup of individuals in a population. Those skilled in the art will be able to synthesize genomic DNA fragments using well known methods.

A population of genomic DNA fragments can have fragments of any desired average length. One skilled in the art will know that fragment size can be chosen based on a variety of factors including, for example, desired resolution, size of the genome, or portion thereof, to be probed, number of genomic DNA fragments in the population of addressed genomic DNA fragments, amount of sequence overlap between overlapping fragments, or the size of gaps between non-overlapping fragments. Resolution is understood to refer to the average length of sequence within which a boundary is determined to reside. Higher resolution correlates with smaller average lengths of sequence within which a boundary is determined to reside. For example, resolution within 2 bases is higher than resolution within 10 or more bases. As described below, the methods can be used in an iterative fashion such that the resolution is increased. For example, a boundary can be identified within 500 or more bases or even 1000 or more bases and subsequent iteration of the methods using a population of smaller fragments covering the identified sequence can improve resolution at which the boundary is identified to within a range of about 100 bases or 50 bases.

One skilled in the art will be able to choose the length of fragments to be used in the methods of the invention according to the desired resolution at which the location of a region or its boundary is to be determined. Specifically, as fragment size is reduced the resolution can be increased. For example, if the size of gaps between or amount of sequence overlap within fragments is held constant and fragment size is reduced the resolution will increase. Correlative with such a decrease in fragment size will be a decrease in the length of genome sequence encoded by the population of fragments. In one embodiment, a genomic DNA fragment of the invention can be at least about 10 kilobases (kb) in length. The methods can be performed with polynucleotide fragments having a shorter length including, for example, those that are at least about 100 bases in length, at least about 500 bases in length, at least about 1 kb in length, at least about 3 kb in length, at least about 5 kb in length, or at least about 8 kb in length. In order to cover longer genomic DNA sequences, larger polynucleotide fragments can also be used in the invention including, for example, those that are at least about 15 kb in length, at least about 20 kb in length, at least about 25 kb in length, at least about 50 kb in length, or at least about 100 kb in length.

The number of genomic DNA fragments in a population can also be chosen based on the desired application of the methods. In cases where resolution requirements are low or the size of the genome being analyzed is small, one can use a small population of fragments. Small populations can be advantageous in reducing computer memory and data processing time, for example, when using the methods to identify pluralities of sequence boundaries. A population of addressed fragments of genomic DNA useful in the invention can include 2 or more addressed genomic DNA fragments. Larger populations can be used to increase resolution at which a relative location of a boundary is to be determined or to cover a larger region of a genome. Accordingly, the methods can be performed with 100 or more fragments, 1×10³ or more fragments, 1×10⁴ or more fragments, 1×10⁵ or more fragments, 1×10⁶ or more fragments, 1×10⁷ or more fragments, 1×10⁸ or more fragments, or 1×10⁹ or more fragments. For many applications of the methods sufficient resolution can be achieved with populations including Thus, the methods can accommodate any desired number of fragments including, for example, 3 or more fragments, 5 or more fragments, 10 or more fragments, 25 or more fragments, 50 or more fragments, 75 or more fragments, 97 or more fragments, 250 or more fragments, or 500 or more fragments.

A population of genomic DNA fragments can contain members having overlapping sequences or non-overlapping sequences. Overlapping sequences can be advantageous for increasing resolution of the methods. The resolution can increase as the redundancy of overlap, or coverage, increases. Specifically, the resolution for a population having fragments with sequences that overlap the sequences of at least two other fragments can be higher than a population in which a sequence of each of the addressed fragments of genomic DNA overlaps a sequence of one other fragment. Further increase in resolution of the methods can be achieved with populations in which most fragments of genomic DNA overlap, for example, at least 3 or more, at least 5 or more, or at least 10 or more other fragments.

A population of genomic DNA fragments having overlapping sequences can be produced by methods that produce fragments of different size or that cleave the DNA at different locations along the sequence. A preparation of genomic DNA can be randomly fragmented, for example, by mechanical shearing, to produce a population of fragments having different size or cleavage locations. A population of genomic DNA fragments having overlapping sequences can also be produced by subjecting a first preparation of genomic DNA to a first fragmentation method, subjecting a second preparation of genomic DNA from the same organism to a second fragmentation method, and using both of the resulting populations of genomic DNA fragments in the methods of the invention. For example, genomic DNA from a single organism or individual can be used to construct separate libraries having different insert sizes, as described for example in Venter et al., supra (2001). Fragments produced by amplification or isolation of the inserts from a first library will overlap with fragments produced by amplification or isolation of different sized inserts from a second library. In another example, separate preparations of genomic DNA can be fragmented with different restriction endonucleases that cleave at different recognition sequences to produce separate populations of fragments in which fragments from one population overlap fragments from a second population.

A population of addressed fragments of genomic DNA having non-overlapping sequences can consist of adjacent fragments of genomic DNA. In addition the fragments can be separated by gaps when aligned to a reference sequence. One skilled in the art will recognize that decreasing size of gaps between fragments will be directly correlated with increased resolution at which a relative location of a boundary can be determined. However, increased size of gaps can allow an increased number of regions to be simultaneously probed. For example, a first population having the same number of similarly sized fragments but larger gaps between fragments when compared to a second population, can be used to cover a larger genome portion and therefore a larger number of genomic regions and their respective boundaries.

The methods of the invention can be used to identify a boundary of a region in a genomic DNA of any size. As described above, a population of fragments can vary in properties such as the size of fragments, number of fragments and amount of sequence overlap between fragments. One skilled in the art will be able to adjust these properties to produce an addressed population of genomic DNA fragments that represent a complete genome or portion thereof. For example, a population of addressed genomic DNA fragments used in the methods of the invention can encode about 100% of a genome sequence or can encode a majority of a genome sequence including, for example, greater than 90% of a genome, greater than 80% of a genome, greater than 70% of a genome or greater than 60% of a genome. A population of addressed genomic DNA fragments used in the methods of the invention can also cover smaller portions of a genome which may be relevant to a specific application including, for example, 5% of a genome, 10% of a genome, 20% of a genome, 30% or a genome, 40% of a genome or 50% of a genome.

A population of addressed fragments of genomic DNA used in the methods of the invention can be attached to any substrate which can be used to distinguish the attached fragment. A substrate to which fragments of genomic DNA are attached and which can be used to distinguish bound fragments from each other includes, for example, a solid phase substrate. The methods of the invention can employ genomic DNA fragments attached to the surface of a solid phase substrate including, for example, a surface of a particle or a surface containing arrayed fragments. A particle having an attached fragment of genomic DNA can be distinguished according to its location relative to other fragment-bound particles in a population. An advantage of using particles in the invention is that a particle can be isolated from reaction conditions with relative ease and washed to remove impurities. Also, particles can be divided and recombined to facilitate manipulations such as combinatorial modifications.

Particles can be fixed at identifiable locations by a variety of methods depending upon the properties of the particle. For example, a particle can be fixed based on size by capturing in a well that has dimensions accommodating a single particle. Accordingly, a population of particles can be fixed in a set of wells that form an array. The size of a particle can also be exploited to capture the particle in a capillary. Thus, multiple particles can be used to form a linear array by capturing the particles in a capillary having a diameter less than the diameter of two particles. Magnetic particles can be used in the methods of the invention and fixed using a magnet.

A solid phase substrates of the invention can be composed of a variety of materials including, for example, paper, glass surface or particle, nitrocellulose, silicon wafer or particle, magnetic bead, agarose or derivatives thereof such as SEPHAROSE™, or polymeric materials such as plastics. A solid phase substrate of the invention can also be modified forms of those described above.

Fragments of genomic DNA can be attached to a substrate such as a solid phase surface via any stable interaction including, for example, affinity interactions, non-specific interactions or covalent interactions. Affinity interactions can be exploited by attaching one of two affinity partners to a polynucleotide fragment and a second affinity partner to a solid phase substrate. Affinity partners useful in the invention include, for example, avidin and biotin, streptavidin and biotin, or an antibody and epitope. Non-specific interactions that can mediate binding of a polynucleotide fragment to a solid phase substrate include, for example, ionic interactions between negatively charged phosphates of the polynulceotide and positively charged groups attached to the Solid phase substrate. Covalent interactions can also be exploited to attach a polynucleotide fragment to a solid phase substrate, for example, using chemical crosslinking methods described below.

The interactions described above for attaching a genomic DNA fragment to a solid phase substrate can be mediated by naturally occurring atoms and moieties of the genomic DNA fragment. For example, nonspecific interactions between the phosphate groups of the polynucleotide fragment and a positively charged group of the solid phase substrate can mediate attachment. A genomic DNA fragment can also be modified to incorporate atoms or molecules that provide attachment capabilities. For example, reactive groups can be added to a genomic DNA fragment to increase reactivity of the genomic DNA fragment with available crosslinking reagents. Accordingly, a genomic DNA fragment can be modified to incorporate reactive moieties such as primary amines, thiols or carbonyls. As described above a modification can also be made to incorporate an affinity group such as a biotin or antibody epitope.

A solid phase substrate can be used directly to attach a genomic DNA fragment or the solid phase substrate can be modified for attachment capability. For example, a solid phase substrate can be chosen based on intrinsic polynucleotide binding properties such as presence of cations. In addition a solid phase substrate can be modified to alter the efficiency or capacity of polynucleotide attachment. For example, a glass surface can be coated with a polycation such as polylysine or polyacrylamide to increase affinity for the phosphate groups of polynucleotide fragments. A solid phase substrate can also be modified to incorporate affinity groups or reactive groups such as those described above for incorporation into polynucleotide fragments. Surface chemistry methods for modification of surfaces or particles are well known in the art and are described, for example, in Pirrung et al., U.S. Pat. No. 5,143,854; Hubbel et al., U.S. Pat. No. 5,571,639; Fodor et al., U.S. Pat. No. 5,744,101; Fodor et al., U.S. Pat. No. 5,489,678; and Winkler et al., U.S. Pat. No. 5,667,195.

A genomic DNA fragment can be covalently attached to a solid phase substrate using a crosslinking reagent. Crosslinking reagents that can be used in the methods of the invention can include, for example, nonspecific reactive groups or reactive groups that are specific for particular atoms or moieties. An example of a non-specific reactive group is a photoreactive group such as an arylazide. Photoreactive groups can be activated by light to form a reactive nitrene or carbene which can nonspecifically form a covalent bond between proximal atoms. Thus, a photoreactive group incorporated in a polynucleotide fragment, a solid phase substrate or a crosslinking reagent can be used to form a covalent bond attaching a polynucleotide fragment to a solid phase substrate. Other non-specific crosslinking reagent that can be used to covalently bond a polynucleotide fragment to a solid phase substrate include, for example, formaldehyde, glutaraldehyde, 4,4′-diazidobiphenyl, or 1,5-diazidonapthalene. Reactive groups that are atom- or moiety-specific can be chosen based on reactive groups present in a polynucleotide fragment or solid phase substrate. Thus, a polynucleotide fragment can be attached to solid supports using homobifunctional and heterobifunctional crosslinkers normally used in protein chemistry. Methods for using such crosslinkers with polynucleotides are described, for example, in Wong, Chemistry of Protein Conjugation and Chemistry, CRC Press (1991).

A genomic DNA fragment used in the methods of the invention can be attached to a solid phase substrate by annealing a portion of the fragment to a complimentary polynucleotide attached to the solid phase substrate. A complimentary polynucleotide attached to the bead can be, for example, a DNA, RNA, or derivative thereof including for example a protein nucleic acid in which the phosphate backbone of the polynucleotide has been replaced with polypeptide linkages. The complimentary polynucleotide can be attached to a solid phase substrate using methods described herein for attaching a genomic DNA fragment.

A genomic DNA fragment can be attached to a defined position on a surface of a solid phase substrate using the attachment methods described above in combination with methods for positional delivery of reagents or positional deposition of polynucleotide fragments. In one embodiment, a polynucleotide fragment can be spotted at a defined position on a surface. An addressed population of polynucleotide fragments can be attached to such a surface by spotting separate polynucleotide samples at discreet locations on the surface such that the spotted areas are separated by a perimeter lacking attached polynucleotide sample. Methods for spatially directed synthesis of polynucleotides are described, for example in Pirrung et al., U.S. Pat. No. 5,143,854; Hubbel et al., U.S. Pat. No. 5,571,639; Fodor et al., U.S. Pat. No. 5,744,101; Fodor et al., U.S. Pat. No. 5,489,678; and Winkler et al., U.S. Pat. No. 5,667,195. Fragments of genomic DNA and/or reagents for their modification can also be delivered to a well in a plate by robotic delivery systems well known in the art.

Following attachment of polynucleotide fragments to a solid phase substrate, unbound polynucleotides can be removed by washing the solid phase substrate. In addition, reactive groups on the polynucleotide and or solid phase substrate can be blocked using appropriate chemistry. Blocking can be achieved by quenching reactive groups or by binding reactive groups to molecules that will be inert to interactions with probe in other steps of the invention. One skilled in the art will know how to quench or block reactive groups according to the reagents used in the attachment step.

A target polynucleotide of the invention can be any polynucleotide that binds a terminal sequence of a region. For example, a boundary of an exon can be determined by the methods of the invention by using a target polynucleotide having a sequence complementary to terminal sequence of an exon, the exon sequence being present in an addressed population of genomic DNA fragments contacted with the target polynucleotide. The methods can be used to determine boundaries for a variety of expressed sequences in a genomic DNA by using an appropriate expressed polynucleotide as a target polynucleotide. Examples of expressed polynucleotides useful as target polynucleotides in the methods include, for example, cDNA, mRNA, ribosomal RNA, tRNA or analogs thereof. A target polynucleotide whether an expressed or other polynucleotide can be isolated from a native cell or synthesized using methods known in the art. Such methods are described for example in Sambrook and Russell, supra and Ausubel, supra.

The methods of the invention can be carried out with a plurality of target polynucleotides thereby providing a population of polynucleotides having different sequences. An example of a plurality of polynucleotides useful in the methods of the invention is a population of expressed sequences from a particular organism, cell or tissue. A plurality of expressed polynucleotides can include all or most of the polynucleotides expressed in an organism, cell or tissue or a subset of the expressed polynucleotides. One skilled in the art will be able to produce pluralities of target polynucleotides with sufficient complexity to include all or most of the expressed sequences in a cell using known methods including, for example, those described in Sambrook and Russell, supra and Ausubel, supra. For example, one skilled in the art will know that in order to have a 99% probability of having each of the estimated 34,000 different mRNA molecules present in a typical mammalian cell, a cDNA based plurality of target polynucleotides would generally contain 5×10⁵ to 1×10⁶ polynucleotides.

The methods of the invention can be used to determine a plurality of sequence boundaries present in expressed sequences from a particular cell or tissue or from a cell exposed to a particular stimulus or set of conditions. For example, the methods can be used to identify sequence boundaries in genomic DNA isolated from a diseased cell such as an aberrantly regulated cell. One skilled in the art can identify a diseased cell from which to obtain target polynucleotides based on properties of the cell indicating the diseased state. For example, an aberrantly regulated cell can be identified according to uncontrolled cell proliferation or altered morphological phenotypes. Specific examples of aberrantly regulated cell types include neoplastic cells such as cancer and hyperplastic cells characteristic of tissue hyperplasia. Another specific example includes immune cells that become aberrantly activated or fail to down regulate following stimulation. Autoimmune diseases are mediated by such aberrantly regulated immune cells. Aberrantly regulated cells can also be identified based on biochemical or physiological dysfunction.

A target polynucleotide can be contacted with an addressed population of genomic DNA fragments under different conditions of stringency. One skilled in the art can readily alter stringency to achieve desired specificity of hybridization. Stringency depends on a variety of factors including, for example, temperature, concentration of genomic DNA fragment and/or target polynucleotide, ionic strength and pH. As known to those of skill in the art, the stability of hybrids is reflected in the melting temperature (Tₘ) of the hybrids. Typically, the hybridization reaction is performed under conditions of lower stringency, followed by washes of varying, but higher, stringency. Reference to hybridization stringency relates to such washing conditions.

Moderately stringent hybridization refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5× SSPE, 0.2% SDS at 42° C., followed by washing in 0.2× SSPE, 0.2% SDS, at 65° C.7

High stringency hybridization refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5× Denhart's solution, 5× SSPE, 0.2% SDS at 42° C., followed by washing in 0.1× SSPE, and 0.1% SDS at 65° C.

Low stringency hybridization refers to conditions equivalent to hybridization in 10% formamide, 5× Denhart's solution, 6× SSPE, 0.2% SDS at 42° C., followed by washing in 1× SSPE, 0.2% SDS, at 50° C. Denhart's solution and SSPE (see, e.g., Sambrook and Russell, supra) are well known to those of skill in the art as are other suitable hybridization buffers.

Therefore, one skilled in the art can contact a target polynucleotide with an addressed population of genomic DNA fragments, under desired conditions of stringency to detect a hybridized sequence. One skilled in the art will know that insufficient signal due to low level hybridization can be increased by decreasing stringency and conversely high levels of background can be reduced by increasing stringency. Thus, the methods of the invention allow for optimization of hybridization conditions to suit the desired application.

A variety of labels can be incorporated into a target polynucleotide to allow detection of a hybridized genomic DNA fragment. Exemplary labels include a radioisotope, a fluorophore, a calorimetric agent, a magnetic substance, an electron-rich material such as a metal, a luminescent tag, an electrochemiluminescent label such as Ru(bpy)₃²⁺, or a binding agent such as biotin. Specific examples of labels for use in detecting nucleic acids are known in the art as described, for example, in the catalogs of Molecular Probes (Eugene, Oreg.) and Synthegen (Houston, Tex.), and in WO 98/59066. Methods for incorporating labels are also well known in the art.

Detection can be achieved by methods specific to the particular label employed. For example, detection of fluorescent probes involves irradiating the probe with an excitatory wavelength of radiation and detecting radiation emitted from the fluorophore by methods known in the art and described for example in Lakowicz, Principles of Fluorescence Spectroscopy, 2^{nd} Ed., Plenum Press New York (1999). Detection of a flourophore can be based on a variety of fluorescence phenomena including, for example, emission wavelength, excitation wavelength, fluorescence resonance energy transfer (FRET), quenching, anisotropy or lifetime. FRET can be used to identify hybridization between a first polynucleotide attached to a donor fluorophore and a second polynucleotide attached to an acceptor fluorophore due to transfer of energy from the excited donor to the acceptor. Thus, hybridization can be detected as a shift in wavelength caused by reduction of donor emission and appearance of acceptor emission for the hybrid compared to the wavelength detected when an excited donor emits radiation due to improper orientation or absence of an acceptor. In addition, fluorescence recovery after photobleaching (FRAP) can be used to identify hybridization according to the increase in fluorescence occurring at a previously photobleached address due to binding of a fluorescently labeled target polynucleotide.

In addition, a hybridized species containing a target polynucleotide and genomic DNA fragment can be detected based on properties of the hybrid including, for example, mass or intrinsic fluorescence. Changes in mass of the hybrid can be detected using any method known in the art for separating and detecting hybrids based on size including, for example, mass spectroscopy techniques. Changes in intrinsic fluorescent properties of a target polynucleotide or genomic DNA fragment can also be detected following hybridization. For example, duplex formation can cause a detectable shift in the excitation or emission wavelength of a nucleic acid as described in Lakowicz, supra.

Detection of target polynucleotide binding in an addressed population of genomic DNA fragments can yield a constellation consisting of a signal readout corresponding to the amount of target polynucleotide bound at each address. As used herein the term “constellation” refers to a set of signals corresponding to the presence or absence of target polynucleotide binding to a plurality of addressed genomic DNA fragments. A constellation can be binary in nature so as to determine presence or absence of bound target polynucleotide at each address in a population or can include intensity of target polynucleotide signal at each address such that an amount of target polynucleotide bound at each address can be determined.

A constellation can be detected by positional scanning of an addressed population or by simultaneous detection of signals from multiple addresses in the population. Positional scanning can include, for example, a series of localized data acquisitions. Each acquisition can be performed to include only a subset of addresses or a single address. A series of localized data acquisitions can be performed to obtain a scan of the solid phase substrate. Scanning can be performed by translating a detection device to different addresses in the population or by translating the addressed population such that individual addresses are detected for each change of position for the population.

Fluorescence detection can be used in a scanning method to detect a constellation of target polynucleotide signals from an addressed population. For example, excitation light can be localized to an address using optical means to limit the area of irradiation. Optical means of focusing an excitation light beam include, for example, use of the objective lense of a microscope.

Simultaneous detection of a constellation of signals can be performed by any method having resolution sufficient to separate signal intensity detected at each address. For example, in cases where a fluorescent probe is used, a charge coupled device (CCD) camera can be used to simultaneously detect intensity and location of a fluorescent emission signal in a constellation.

Fragments of genomic DNA can also be detected by optical mapping techniques as described, for example, in Lin et al., Science 285:1558-1562 (1999). Briefly, optical mapping is a method that can be used to construct an ordered map of a genomic DNA from fragments of the genomic DNA. The fragments can be labeled using methods described above, for example, by a fluorophore and attached to a solid phase also as described above. The fragments can be imaged by a system appropriate to the particular label used, for example, an optical imaging system, such as those commonly available in a light microscope, can be used to image fluorescently labeled fragments. In one embodiment, addressed fragments can be imaged by a semiautomated image acquisition system that collects successive images and correctly assembles them into one superimage including, for example, Visionade as described in Lin et al., supra (1999). Following imaging, contigs covering the genomic DNA can be assembled manually or by a known algorithm that automatically computes contigs of a genomic map such as the Gentig algorithm described in Lin et al., supra (1999). Once the contigs are mapped, boundary regions can be identified using methods described below.

In cases where a relationship can be established between intensity of signal detected and quantity of hybridized target polynucleotide, the methods can be used to quantitate the amount of target polynucleotide hybridized at each address. Therefore, in the methods of the invention, a step involving identifying a constellation of genomic DNA fragments hybridized to the target polynucleotide can include identifying a constellation consisting of an amount of target polynucleotide hybridized at one or more addresses.

In one embodiment of the invention 2 or more labels can be used simultaneously. Detection of the labels can be performed simultaneously so long as signals from the two target polynucleotides can be distinguished. For example, 2 fluorophores can be simultaneously detected by measuring emission intensity at 2 wavelengths where the emission spectra of the fluorophores do not significantly overlap. Additionally, labels can be simultaneously present in a constellation and detected separately. For example, a population of target polynucleotides containing 2 fluorophores having non-overlapping excitation wavelengths can be irradiated twice, once for each flourophore, to provide individual detection of the two labels. In the latter case a temporal separation of excitation/detection events can be used to distinguish target polynucleotides even in cases where the emission from two or more fluorophores can not be conveniently distinguished.

The methods of the invention provide for optimization of detection by altering detection sensitivity. One skilled in the art will be able to alter detection sensitivity according to the particular technique used for detection using, for example, guidance provided in the references disclosed above.

A genomic DNA fragment that specifically binds to a target polynucleotide can be identified as having a sequence complementary to the target polynucleotide. Thus, for a target polynucleotide that has a sequence complementary to a terminal sequence of a region, the bound fragment can be identified as potentially having the terminal sequence of the region. The location of the terminal sequence in the genomic DNA fragment can be identified by obtaining a sequence of the fragment and orienting the fragment with respect to other fragments in the population by aligning the sequences of the fragments with the sequence of the genomic DNA. The sequence of a fragment in the addressed population can be obtained using well known methods for nucleotide sequence analysis including, for example, automated sequencing instruments known in the art, or other methods based on Maxam-Gilbert or Sanger methods of sequencing as described in Sambrook and Russell, supra and Ausubel, supra. Alternatively, a sequence can be obtained from prior knowledge of the sequence of the fragment due to indexing of the addressed population. An index of an addressed population includes determination of the sequence for genomic DNA fragments at particular addresses in the population based on sequencing or on a reference sequence used to direct synthesis of the fragment. An index can include sequences for all or a subset of the genomic DNA fragments present in the addressed population and can represent full or partial sequences for each genomic DNA fragment.

Once the sequence is obtained it can be used to orient fragments with respect to each other in the genomic DNA sequence. Oriented fragments can be compared to the binding pattern observed between fragments and target polynucleotide. Oriented fragments having adjacent or overlapping sequences that alternatively bind a target polynucleotide can be identified as having sequences that flank a boundary of a terminal sequence of the target polynucleotide. Thus, the boundary can be identified relative to a sequence in one or both of the alternatively bound genomic DNA fragments. As described above, the resolution at which a location of the boundary can be determined will depend upon properties of the genomic DNA fragments including, for example, length of the fragments, amount of sequence overlap between overlapping fragments or amount of sequence between non-overlapping fragments.

The methods can be performed in an iterative fashion to increase resolution at which a boundary is identified. Specifically, once a length of sequence has been identified in the methods of the invention to have a sequence boundary, a new addressed population of genomic DNA fragments can be produced such that the fragments cover the identified sequence with higher resolution. Such a population, when compared to the population used in the previous iteration of the method can have, for example, shorter length, greater amount of sequence overlap between overlapping fragments or reduced amount of sequence between non-overlapping fragments. The methods can then be repeated by contacting the new addressed population with the target polynucleotide used in the previous iteration. Accordingly, the location of the boundary can be identified at higher resolution.

The resolution can be further augmented by empirically determining the location where a target polynucleotide binds a particular genomic DNA fragment. For example, a genomic DNA fragment identified by the methods of the invention as having a terminal sequence of a target polynucleotide can be contacted with the target polynucleotide and the location of the terminal sequence identified by a nuclease protection assay. Specifically, a nuclease that preferentially digests single stranded portions of polynucleotides compared to double stranded portions can be used to remove unbound sequence from the complex. Following digestion, the sequence of the undigested portion of the genomic DNA fragment can be determined and its terminus identified. The sequence boundary can then be identified as being adjacent to the terminus. Alternatively, a terminal sequence can be identified using a primer extension method in which a polymerase or reverse transcriptase extends a labeled target polynucleotide using a bound genomic DNA fragment as template. Such nuclease protection and primer extension assays can be performed using methods known in the art as described in Sambrook and Russell, supra and Ausubel, supra.

A coding sequence flanking a sequence boundary identified by the methods of the invention can be used in a gene identification method to identify another genomic DNA sequence that codes for the same protein including, for example, an additional exon. Thus, a boundary sequence identified by the methods of the invention can be used to determine an mRNA or protein sequence. A gene identification method can include, for example, synthesis of a nucleic acid primer according to the identified sequence and probing a library or second population of expressed polynucleotides with the primer. An expressed polynucleotide determined to hybridize with the primer can then be isolated and identified by sequencing. Alternatively, a sequence identified by the methods of the invention can be used as an input or query sequence in a gene identification algorithm capable of determining a gene to which the query sequence belongs or predicting gene sequences based on the query sequence. Gene identification algorithms are known in the art as described, for example, in Sze et al., Bioinformatics 14:14-19 (1998); Mironov et al., Genomics 51:332-339 (1998), and Arslan et al., Bioinformatics 17:327-337 (2001).

An expressed sequence, or boundary thereof, identified by the methods of the invention can be translated into a polypeptide sequence and the polypeptide sequence used to search a protein database. A protein database can be searched to identify a protein encoded by an identified sequence or to identify other proteins having similar sequence. A protein database can be searched using BLAST, Basic Local Alignment Search Tool, which can be used according to default parameters as described by Tatiana et al., FEMS Microbial Lett. 174:247-250 (1999) or on the National Center for Biotechnology Information web page at ncbi.nlm.gov/BLAST/. BLAST is a set of similarity search programs designed to examine all available sequence databases and can function to search for similarities in amino acid or nucleic acid sequences. A BLAST search provides search scores that have a well-defined statistical interpretation. Furthermore, BLAST uses a heuristic algorithm that seeks local alignments and is therefore able to detect relationships among sequences which share only isolated regions of similarity including, for example, protein domains (Altschul et al., J. Mol. Biol. 215:403-410 (1990)).

In addition to the originally described BLAST (Altschul et al., supra, 1990), modifications to the algorithm have been made (Altschul et al., Nucleic Acids Res. 25:3389-3402 (1997)). One modification is Gapped BLAST, which allows gaps, either insertions or deletions, to be introduced into alignments. Allowing gaps in alignments tends to reflect biologic relationships more closely. For example, gapped BLAST can be used to identify sequence identity within similar domains of two or more proteins. A second modification is PSI-BLAST, which is a sensitive way to search for sequence homologs. PSI-BLAST performs an initial Gapped BLAST search and uses information from any significant alignments to construct a position-specific score matrix, which replaces the query sequence for the next round of database searching. A PSI-BLAST search is often more sensitive to weak but biologically relevant sequence similarities.

A second resource that can be used to identify a protein encoded by an identified sequence or to identify other proteins having similar sequence is PROSITE, available on the world wide web at ExPASy. PROSITE is a method of determining the function of uncharacterized proteins translated from genomic or cDNA sequences (Bairoch et al., Nucleic Acids Res. 25:217-221 (1997)). PROSITE consists of a database of biologically significant sites and patterns that can be used to identify which known family of proteins, if any, a query sequence belongs. In some cases, the sequence of an unknown protein is too distantly related to any protein of known structure to detect similarity by overall sequence alignment. However, a protein that is substantially the same as another protein can be identified by the occurrence in its sequence of a particular cluster of amino acid residues, which can be called a pattern, motif, signature or fingerprint, that is substantially the same as a particular cluster of amino acid residues in the other protein including, for example, those found in similar domains. PROSITE uses a computer algorithm to search for motifs that identify proteins as family members. PROSITE also maintains a compilation of previously identified motifs, which can be used to determine if a newly identified protein is a member of a known protein family.

The methods of the invention can be used as an alternative to searching a database of expressed sequence tags (ESTs). EST data provide a tool for identifying transcribed sequences. However, EST databases are generally incomplete with respect to coverage of all exons in a particular gene since ESTs are generally shorter than a full length gene. As described above, the methods of the invention can be used with a population of genomic DNA fragments covering an entire genome. Such a population can be probed with a target polynucleotide representing an expressed sequence to identify the location of a boundary for the expressed sequence. Thus, a boundary for one or more exon encoding the expressed sequence can be identified. An addressed population of genomic DNA fragments that is indexed relative to the full length genomic DNA can be used to rapidly and conveniently identify one or more exon boundaries for a single expressed sequence or for multiple sequences.

The methods of the invention can be used to determine differential parsing of regions in two or more polynucleotides correlating, for example, with addition, deletion or relocation of a region in a first polynucleotide compared to a second polynucleotide. Specifically, the methods can be performed using the same population of addressed genomic DNA fragments with a first target polynucleotide and a second target polynucleotide. The location of sequence boundaries determined for the two target polynucleotides can be compared to determine differential parsing, also referred to in the art as differential expression. The methods can be used to determine differential parsing for a variety of target polynucleotides including, for example, polynucleotides expressed in different cells or tissues of the same organism, polynucleotides expressed by the same cell in response to different conditions or stimuli, or polynucleotides from different individuals. Additionally, a comparison can be made between a sequence boundary determined by the methods of the invention and a sequence boundary identified by any other method in order to identify differential parsing.

The invention further provides a tissue specific array, including a population of addressed genomic DNA fragments encoding specifically expressed sequences of the genomic DNA. A specifically expressed sequence refers to a sequence of a polynucleotide that is transcribed from a genomic DNA by a specific cell, tissue, or organism in a particular environment or condition. A tissue specific array can preferentially include sequences that are specifically expressed. Thus, the array can be produced such that non-expressed sequences are omitted. An advantage of a population of addressed genomic DNA fragments encoding specifically expressed sequences of the genomic DNA is that the amount of reagents and computational processing time required to identify expression patterns in an individual are reduced.

In one embodiment, a tissue specific array can preferentially include exons. For example, a tissue specific population of addressed genomic DNA fragments can encode a plurality of exons expressed in the tissue. An exon encoded by a tissue specific population can be encoded by other tissues of the same organism or can be uniquely expressed in the tissue. Additionally, the order of the exons in a particular expressed sequence can be unique to the tissue. Thus, a tissue specific array can be useful for rapid and efficient determination of presence or absence of expression for a particular exon or determination of the particular pattern of exons in an expressed sequence as affected, for example, by differential splicing.

A population of addressed fragments encoding specifically expressed sequences of a genomic DNA can be used to rapidly and efficiently monitor expression differences between individuals or in response to different conditions. For example, a population of fragments encoding exons expressed by a tissue can be used to screen for expression changes due to various stimuli to the tissue including, for example, exposure to a compound such as a therapeutic drug. The expression changes can be prognostic or diagnostic of a therapeutic effect for a drug or can indicate an adverse effect of the drug. Additionally, a population of fragments encoding exons expressed by an individual can be used to determine a mutation or polymorphism when used to probe target polynucleotides encoding exons expressed by other individuals. Depending upon the extent to which the population of fragments covers the genome of the tissue or individual in the above-described examples, the population can be used to determine a global response to a drug or to simultaneously determine polymorphisms or mutations at a large number of loci.

It is understood that modifications which do not substantially affect the activity of the various embodiments of this invention are also included within the definition of the invention provided herein. Accordingly, the following examples are intended to illustrate but not limit the present invention.

Throughout this application various publications have been referenced. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A method for determining a sequence boundary, comprising:
(a) contacting a population of addressed fragments of eukaryotic genomic DNA with a target polynucleotide, said target polynucleotide binding a terminal sequence of a DNA region, said addressed fragments of eukaryotic genomic DNA being at least 100 nucleotides in length;
(b) determining a relative order for 2 or more of said addressed fragments compared to a sequence of said genomic DNA;
(c) identifying a pair of fragments among said 2 or more addressed fragments that alternatively bind said terminal sequence of a region; and
(d) determining for said sequence of said genomic DNA a relative location of a boundary of said region compared to a location of at least one genomic DNA fragment in said pair.

2. The method of claim 1, wherein said boundary separates an exon from an intron.

3. The method of claim 1, wherein said target polynucleotide comprises cDNA.

4. The method of claim 1, wherein said target polynucleotide comprises RNA.

5. The method of claim 1, wherein said addressed fragments of eukaryotic genomic DNA are surface bound.

6. The method of claim 5, wherein said surface is a particle.

7. The method of claim 5, wherein said surface is a location on an array.

8. The method of claim 1, wherein said pair of fragments have a portion of overlapping sequence.

9. The method of claim 1, wherein said pair of fragments have a portion of adjacent sequence compared to said sequence of said genomic DNA.

10. The method of claim 1, wherein said addressed fragments of eukaryotic genomic DNA are at least 200 nucleotides in length.

11. The method of claim 1, wherein said addressed fragments of eukaryotic genomic DNA are at least 500 nucleotides in length.

12. The method of claim 1, wherein said addressed fragments of eukaryotic genomic DNA are at least 1000 nucleotides in length.

13. A method for determining a sequence boundary, comprising:
(a) contacting a population of surface-bound fragments of eukaryotic genomic DNA with a target polynucleotide, said target polynucleotide binding a terminal sequence of a DNA region, said addressed fragments of eukaryotic genomic DNA being at least 100 nucleotides in length;
(b) determining a relative order for 2 or more of said surface-bound fragments compared to a sequence of said genomic DNA;
(c) identifying a pair of fragments among said 2 or more surface-bound fragments that alternatively bind said terminal sequence of a region; and
(d) determining for said sequence of said genomic DNA a relative location of a boundary of said region compared to a location of at least one genomic DNA fragment in said pair.

14. The method of claim 13, wherein said boundary separates an exon from an intron.

15. The method of claim 13, wherein said target polynucleotide comprises cDNA.

16. The method of claim 13, wherein said target polynucleotide comprises RNA.

17. The method of claim 13, wherein said addressed fragments of eukaryotic genomic DNA are surface bound.

18. The method of claim 17, wherein said surface is a particle.

19. The method of claim 17, wherein said surface is a location on an array.

20. The method of claim 13, wherein said pair of fragments have a portion of overlapping sequence.

21. The method of claim 13, wherein said pair of fragments have a portion of adjacent sequence compared to said sequence of said genomic DNA.

22. The method of claim 13, wherein said addressed fragments of eukaryotic genomic DNA are at least 200 nucleotides in length.

23. The method of claim 13, wherein said addressed fragments of eukaryotic genomic DNA are at least 500 nucleotides in length.

24. The method of claim 13, wherein said addressed fragments of eukaryotic genomic DNA are at least 1000 nucleotides in length.

25. A method for determining a plurality of sequence boundaries, comprising:
(a) contacting a population of addressed fragments of eukaryotic genomic DNA with a target polynucleotide, said target polynucleotide binding a plurality of terminal sequences of DNA regions, said addressed fragments of eukaryotic genomic DNA being at least 100 nucleotides in length;
(b) determining a relative order for 2 or more of said addressed fragments compared to a sequence of said genomic DNA for a plurality of sets of 2 or more genomic DNA fragments;
(c) identifying a plurality of pairs of fragments among said plurality of sets of 2 or more addressed fragments, said pairs comprising fragments that alternatively bind said terminal sequences of regions; and
(d) determining for said sequence of said genomic DNA relative locations of boundaries for a plurality of said regions compared to locations of at least one genomic DNA fragment in each of said pairs.

26. The method of claim 25, wherein said boundaries separate exons from intron.

27. The method of claim 25, wherein said target polynucleotide comprises cDNA.

28. The method of claim 25, wherein said target polynucleotide comprises RNA.

29. The method of claim 25, wherein said addressed fragments of eukaryotic genomic DNA are surface bound.

30. The method of claim 29, wherein said surface is a particle.

31. The method of claim 29, wherein said surface is a location on an array.

32. The method of claim 25, wherein said pairs of fragments have portions of overlapping sequence.

33. The method of claim 25, wherein said pairs of fragments have portions of adjacent sequence compared to said sequence of said genomic DNA.

34. The method of claim 25, wherein said addressed fragments of eukaryotic genomic DNA are at least 200 nucleotides in length.

35. The method of claim 25, wherein said addressed fragments of eukaryotic genomic DNA are at least 500 nucleotides in length.

36. The method of claim 25, wherein said addressed fragments of eukaryotic genomic DNA are at least 1000 nucleotides in length.
